(19) United States
(12) Patent Application Publication
(10) Pub. No.: US 2020/0240983 A1
(43) Pub. Date: Jul. 30, 2020

(54) CAPACITIVE MICRO-SENSOR FOR PATHOGEN-SPECIFIC ANTIBODY RESPONSES

(71) Applicant: Colorado State University Research Foundation, Fort Collins, CO (US)

(72) Inventors: Lei Wang, Fort Collins, CO (US); Jessica Filer, Fort Collins, CO (US); David Dandy, Fort Collins, CO (US); Brian Geiss, Fort Collins, CO (US)

(73) Assignee: Colorado State University Research Foundation, Fort Collins, CO (US)

(21) Appl. No.: 16/746,177

(22) Filed: Jan. 17, 2020

Related U.S. Application Data
(60) Provisional application No. 62/796,647, filed on Jan. 25, 2019.

(51) Int. Cl. G01N 33/543; G01N 27/327; G01N 33/569

(52) U.S. Cl. CPC G01N 33/5438; G01N 33/569; G01N 27/3275

(57) ABSTRACT

A novel technique for label-free, rapid detection of ultra-low concentrations of virus specific antibodies is described. We have developed a simple, robust capacitive biosensor using microwires coated with Zika or Chikungunya virus envelope antigen. With little discernable nonspecific binding, the sensor can detect as few as 10 antibody molecules in a small volume (10 molecules/30 μL) within minutes. It can also be used to rapidly, specifically, and accurately determine the isotype of antigen-specific antibodies. Finally, we demonstrate that anti-Zika virus antibody can be sensitively and specifically detected in dilute mouse serum and can be isotyped using the sensor. Overall, our findings indicate that our microwire sensor platform can be used as a reliable, sensitive, and inexpensive diagnostic tool to detect immune responses at the point of care.

### RELATED APPLICATIONS

This application claims priority under 35 U.S.C. § 119(e) to U.S. Provisional Patent Application No. 62/796,647, filed Jan. 25, 2019, which is incorporated herein by reference.

### GOVERNMENT SUPPORT

This invention was made with government support under Grant Nos. RO1 AI114675 and RO1 AI132668 awarded by the National Institutes of Health and 1332404 and 1450032 from the National Science Foundation. The government has certain rights in the invention.

### BACKGROUND OF THE INVENTION

Analyzing the humoral antibody response in clinical samples is critical to diagnose infectious disease, understand pathogenesis and immune response kinetics, and develop vaccines, and the enzyme-linked immunosorbent assay (ELISA) is used as the gold standard clinical diagnostic tool for antibody detection. However, ELISAs require large instrumentation in centralized laboratories and specialized training to execute and interpret the results which limits the utility of ELISAs in low-resource settings. Many cases therefore go undiagnosed which indicates an urgent need for sensitive, robust assays that quickly diagnose infection at point of care (POC) and provide health-care providers with actionable information. While lateral flow assays are promising candidates for POC applications, these assays often lack sensitivity and demonstrate interference from matrix components of unprocessed samples.

Capacitive biosensors employ direct sample application for label-free detection. Other electrochemical antibody sensors have been developed for serological analysis, but these designs incorporate enzymatic labels or redox couples that increase complexity and cost. Compared to other immunosensors, capacitive biosensors are ideal candidates for sensitive and label-free bioanalysis platforms. Capacitive sensing is based on the theory of the electrical double layer (DL), where the working electrode is conjugated with probe that binds a target to increase the length of the DL. Because capacitance is inversely proportional to the DL length, this increase produces a corresponding decrease in capacitance. Such capacitive signals provide a direct, rapid measure of target binding. Based on our previous work using capacitance to detect DNA (Biosens. Bioelectron. 2016, 87, 646), the sensitivity of capacitive biosensors is far superior to traditional diagnostic assays and is ideal to detect low antibody titers during early stages of infection. Capacitive biosensors are thus an attractive sensing modality that has not yet been fully explored for specific antibody detection.

Resource intensive diagnostic tools limits their utility of for point of care service. Accordingly, there is a need for alternate technologies for a POC platform that can specifically detect low levels of antibodies in serum.

### SUMMARY

This disclosure provides a capacitive immunosensor that specifically detects ZIKV and Chikungunya (CHIKV) antibodies using a sensor modified with their respective envelope (E) protein. It directly measures monoclonal antibody with a lower boundary of approximately 10 antibody molecules in a 30 μL sample. The antibody detection system discriminates between antibodies with little cross-reactivity and can even differentiate isotypes, indicating marked selectivity. We also demonstrate that our system can specifically and sensitively detect polyclonal anti-ZIKV antibodies present in mouse serum. This method is distinguished from previous antibody detection methods not only in the platform, but also by its superior sensitivity and specificity.

Accordingly, this disclosure provides a micro-sensor comprising:
a) a working electrode covalently bonded to head-groups of a self-assembled monolayer (SAM), wherein the SAM comprises alkyl chains, wherein the alkyl chains are substituted at one end with a head-group and functionalized at a terminal end with a functional group; and
b) pathogen-specific antigens bioconjugated to at least 10% of the functional groups of the SAM;

wherein the micro-sensor is label-free and changes in electrical properties of the working electrode are detectable when an antibody binds with specificity to the antigen and forms an antigen-antibody complex.

This disclosure also provides a method forming a micro-sensor comprising:
a) contacting a noble metal and a mixture of HS(C₃-C₃₀)alkyl-OH and HS(C₃-C₃₀)alkyl-CO₂H to form a self-assembled monolayer (SAM) covalently bonded to the surface of the noble metal via the sulfur moieties in the mixture;
b) bioconjugating pathogen-specific antigens of a virus envelope protein to —CO₂H moieties of SAM, thereby forming a working electrode; and
c) spacing a reference electrode adjacent to the working electrode thereby forming the micro-sensor;

wherein the micro-sensor is label-free and changes in electrical properties of the working electrode are detectable when an antibody binds with specificity to the antigen and forms an antigen-antibody complex.

Additionally, this disclosure provides a method for detecting antibodies comprising:
a) contacting a sample with a micro-sensor, wherein the micro-sensor comprises:
   i) a gold working electrode covalently bonded to sulfur atoms of a self-assembled monolayer (SAM), wherein the SAM comprises —(C₃-C₃₀)alkyl-chains substituted at one end with sulfur and functionalized at a terminal end with a functional group;
   ii) pathogen-specific antigens of a virus envelope protein bioconjugated to at least 10% of the functional groups of the SAM; and
   iii) a reference electrode; and
b) determining the presence or absence of a change in capacitance of the microsensor;

wherein the micro-sensor is label-free and changes in capacitance relative to the reference electrode are detectable when an antibody that is present in the sample binds with specificity to the antigen of the working electrode and forms an antigen-antibody complex.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Schematic of capacitive immunosensor design and working principles. (a) Device layers and resulting immunosensor shown from the top. RE: reference electrode, WE: working electrode; (b) Working electrode (Au microwire) surface chemistry and functionalized layers, with the corresponding equivalent circuit and total capacitance equation. DL capacitance, C_{DL}, is placed in parallel with a leakage resistance, Rₗₑₐₖ. C_{DL} represents the total capacitance, Cₜₒₜ, of the individual capacitance contribution from each surface layer.

FIG. 2. Specificity tests with monoclonal antibodies. a) Illustration of ZIKV E antigen as the recognition element to test one specific and three nonspecific antibodies; (b) Capacitance responses for the four antibodies at concentrations from 0 to 10³ molecules per 30 μL in 1×PBST buffer (n=3 at each concentration, mean±STD). The regression fit for specific anti-ZIKV E is shown in the plot as a dashed line.

FIG. 3. Isotyping tests with monoclonal antibodies. (a) Illustration of CHIKV E antigen-antibody complex to determine the isotype of anti-CHIKV E (IgG 2b). Six secondary antibodies are used here to perform the test: anti-IgG1, anti-IgG2a, anti-IgG2b, anti-IgG3, anti-IgA and anti-IgM; (b) Capacitance responses of the isotype tests with six secondary antibodies at concentrations from 0 to 10³ molecules per 30 μL in 1×PBST buffer (n=3 at each concentration, mean±STD). A regression fit is shown in the plot for secondary IgG2b antibody. Similar results were obtained for IgG isotyping of anti-ZIKV monoclonal antibody (FIG. 8).

FIG. 4. Immune response kinetics for mouse serum samples. Capacitive response to mouse serum at different time points pre-and-post vaccination with ZIKV. (a) Mouse serum tested at a 1:10¹² dilution in 1×PBST buffer; (b) Mouse serum tested at a 1:10⁶ dilution in 1×PBST buffer. Three biological samples (n=3, mean±STD) for each time point were tested except for Day 14 (n=2, mean±STD). Each biological sample shown is the average of three technique replicates. A paired t-test was carried out between pre- and post-vaccination with ZIKV samples. * paired t-test: p<0.05.

FIG. 5. Isotyping of anti-ZIKV antibodies in mouse serum samples. Capacitive response of antibody isotypes in mouse serum at Day 4 and 21 with ZIKV. Mouse serum was used at a 1:10⁶ dilution in 1×PBST buffer to saturate the surface for isotype detection. Three biological samples (n=3, mean±STD) for each time point were tested. Each biological sample shown is the average of three technical replicates.

FIG. 6. Western blot analysis of IgG antibody responses in mice immunized with Zika DNA vaccine. * denotes the presence of anti-Zika envelope reactivity in Day 21 samples.

FIG. 7. (a) Illustration of CHIKV E antigen as the recognition element to test one specific and three nonspecific antibodies; (b) Capacitance responses for four antibodies at concentrations from 0 to 103 molecules per 30 μL in 1×PBST buffer (n=3 at each concentration, mean±STD). The fit for specific anti-CHIKV E is shown in the plot as a dashed line.

FIG. 8. (a) Illustration of ZIKV E antigen-antibody complex to determine the isotype of anti-ZIKV E (IgG 2b). Three secondary antibodies are used here to perform the test: anti-IgG and anti-IgM; (b) Capacitance responses of the isotype tests with IgG or IgM secondary antibodies at concentrations from 0 to 10³ molecules per 30 μL in 1×PBST buffer (n=3 at each concentration, mean±STD). A regression fit is shown in the plot for secondary IgG antibody.

FIG. 9. Specificity tests with mouse serum samples. (a) The difference between the negative capacitance change for Day 21 and pre-immune mouse serum samples at a 1:10¹² dilution in 1×PBST buffer are compared for ZIKV E and CHIKV E recognition antigens (n=3 at each concentration, mean±STD). (b) The difference between the negative capacitance for Day 21 and pre-immune mouse serum samples at a 1:10⁶ dilution in 1×PBST buffer are compared for ZIKV E and CHIKV E recognition antigens (n=3 at each concentration, mean±STD). ** paired t-test: p<0.01.

FIG. 10. Capacitive responses of pre-immune and Day 4 after ZIKV infected mouse serums at a wide range of dilutions from 1:10¹⁸ to 1:10³ dilutions in 1×PBST buffer (n=3 at each dilution).

FIG. 11. ELISA analysis of anti-Zika IgM and IgG levels in Mice 3, 4, and 6. 1:100 dilutions of serum were used to test each sample.

FIG. 12. Schematics of gold microwire surface modification, probe immobilization (left image), blocking and target binding (right image).

FIG. 13. Dependence of electron transfer resistance change (Ret SAM—Ret probe) on probe (ZIKV E) incubation time.

FIG. 14. The plot of the electron transfer resistance (Ret) of each step after gold microwire treatments on ePAD. ** paired t-test: p<0.01; * paired t-test: p<0.05; N. S. not significant.

FIG. 15. Changed electron transfer resistance vs logarithm of concentrations of the specific (ZIKV E mAb) and nonspecific (M13 mAb) targets.

### DETAILED DESCRIPTION

Detection of viral infection is commonly performed using serological techniques like the enzyme-linked immunosorbent assay (ELISA) to detect antibody responses. Such assays may also be used to determine the infection phase based on isotype prevalence. However, ELISAs demonstrate limited sensitivity and are difficult to perform at the point of care.

The goal of this work is to develop a novel POC platform that can specifically detect low levels of antibodies in serum. Due to its clinical relevance, Zika virus (ZIKV) was chosen as a model system to validate the platform. ZIKV is an emerging Flavivirus that is closely related to other mosquito-borne viruses like yellow fever, West Nile, and dengue. It recently became a major public health concern due to neurological complications in infected adults and severe developmental complications for fetuses of infected women. Therefore, accurate and early diagnosis of ZIKV is essential for proper monitoring and medical intervention in these cases.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2, 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

An “effective amount” refers to an amount effective to bring about a recited effect, such as an amount necessary to form products in a reaction mixture. Determination of an effective amount is typically within the capacity of persons skilled in the art, especially in light of the detailed disclosure provided herein. The term “effective amount” is intended to include an amount of a compound or reagent described herein, or an amount of a combination of compounds or reagents described herein, e.g., that is effective to form products in a reaction mixture. Thus, an “effective amount” generally means an amount that provides the desired effect.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983).

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, N.Y., 1994), and references cited therein); and Comprehensive Organic Transformations, Larock, R. C., Second Edition, John Wiley & Sons, New York (1999), and referenced cited therein.

As used herein, the term “substituted” or “substituent” is intended to indicate that one or more (for example, 1-20 in various embodiments, 1-10 in other embodiments, 1, 2, 3, 4, or 5; in some embodiments 1, 2, or 3; and in other embodiments 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described herein. The alkyl can also be optionally partially or fully unsaturated. As such, the recitation of an alkyl group can include both alkenyl and alkynyl groups. The alkyl can be a monovalent hydrocarbon radical, as described and exemplified above, or it can be a divalent hydrocarbon radical (i.e., an alkylene).

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like. The cycloalkyl can be unsubstituted or substituted.

The term “noble metal” refers to, for example, ruthenium, rhodium, palladium, silver, osmium, iridium, platinum, or gold.

The term “self-assembled monolayers (SAM)” refers to organic molecules that are molecular assemblies formed spontaneously on surfaces by adsorption and are organized into ordered domains. SAMs are created by the chemisorption of “head groups” onto a substrate from the liquid phase followed by a slow organization of “tail groups”. The tail groups can have an organic functional group at the terminal end or the end opposite head groups. Typically, head groups are connected to a molecular chain in which the terminal end can be functionalized (i.e. adding —OH, —NH₂, —COOH, or —SH groups) to vary the interfacial properties.

Self-assembly is a process in which a disordered system of pre-existing components forms an organized structure or pattern because of specific, local interactions among the components themselves, without external direction. For molecular self-assembly, initially, at small molecular density on the surface, adsorbate molecules form either a disordered mass of molecules or form an ordered two-dimensional “lying down phase”, and at higher molecular coverage, over a period of minutes to hours, begin to form three-dimensional crystalline or semi-crystalline structures on the substrate surface. The “head groups” assemble together on the substrate, while the tail groups assemble far from the substrate. Areas of close-packed molecules nucleate and grow until the surface of the substrate is covered in a single monolayer.

### Embodiments of the Invention

This disclosure provides a micro-sensor comprising:
a) a working electrode covalently bonded to head-groups of a self-assembled monolayer (SAM), wherein the SAM comprises alkyl chains, wherein the alkyl chains are substituted at one end with a head-group and functionalized at a terminal end with a functional group; and
b) pathogen-specific antigens bioconjugated to at least 10% of the functional groups of the SAM;

wherein the micro-sensor is label-free and changes in electrical properties of the working electrode are detectable when an antibody binds with specificity to the antigen and forms an antigen-antibody complex.

In various embodiments, the pathogen-specific antigen comprises an antigen of a virus envelope protein. In other various embodiments, the pathogen-specific antigen comprises an antigen of a flavivirus envelope protein or an alphavirus envelope protein. In additional various embodiments, the pathogen-specific antigen comprises an antigen of envelope proteins of Chikungunya virus, Zika virus, Dengue virus, Yellow fever virus, or West Nile virus.

In other embodiments, the pathogen-specific antigen comprises an antigen of Dengue fever, Hepatitis C, Japanese encephalitis, Kyasanur Forest disease, Murray Valley encephalitis, St. Louis encephalitis, Tick-borne encephalitis, West Nile encephalitis, Yellow fever, or Zika fever. In yet other embodiments, the pathogen-specific antigen comprises an antigen of a Barmah Forest virus complex, Eastern equine encephalitis complex, Middelburg virus complex, Ndumu virus complex, Semliki Forest virus complex, Venezuelan equine encephalitis complex, or Western equine encephalitis complex.

In various additional embodiments, the pathogen specific antigen comprises the antigen from a DNA virus such as herpesviruses, poxviruses, hepadnaviruses, asfarviridae, adenoviridae, or papillomaviridae. In other additional embodiments, the pathogen specific antigen comprises the antigen from an RNA virus such as flavivirus, alphavirus, togavirus, coronavirus, hepatitis d, orthomyxovirus, paramyxovirus, rhabdovirus, bunyavirus, filovirus, picornaviridae, or caliciviridae. In other additional embodiments, the pathogen specific antigen comprises the antigen from a retrovirus or bacteria.

In various embodiments, the pathogen-specific antigens bioconjugated to at least 20% of the functional groups of the SAM, at least 40% of the functional groups of the SAM, or at least 60% of the functional groups of the SAM. In various other embodiments, the micro-sensor is configured to detect changes in capacitance when an antibody binds with specificity to the antigen and forms an antigen-antibody complex.

In further embodiments, the pathogen-specific antigen is bioconjugated to the functional groups via an amide bond. In additional embodiments, the SAM comprises a second functional group comprising a hydroxyl. In other embodiments, the alkyl chains comprise —(C₃-C₃₀)alkyl-, —(C₄-C₃₀)alkyl-, —(C₅-C₃₀)alkyl-, —(C₆-C₃₀)alkyl-, or —(C₆-C₂₀)alkyl-. In yet other embodiments, the head groups comprise sulfur, silicon, phosphorous, germanium, selenium, aluminum, oxygen, nitrogen, tin, or lead. In some other embodiments, the working electrode comprises a metal or a metal etched with plasma, for example a noble metal etched with oxygen.

In additional embodiments, the micro-sensor comprises a silver microwire reference electrode, wherein the working electrode comprises a gold microwire, the alkyl chains are —S(C₃-C₃₀)alkyl-X covalently bonded to the gold microwire via the sulfur atom of —S(C₃-C₃₀)alkyl-X, wherein X of about 30% to about 80% (or X of about 40% to about 60%, or X of about 45% to about 55%) of the alkyl chains is a bioconjugated pathogen-specific antigen, such as a flavivirus envelope protein, and X of the remaining percentage of the alkyl chains is a functional group comprising hydroxyl, carboxyl, or amide. In other embodiments, SAM further comprises —S(C₃-C₃₀)alkyl-X wherein X is different or is selected from the group consisting of hydroxyl, carboxyl, or amide.

This disclosure also provides a method for forming a micro-sensor comprising:
a) contacting a noble metal and a mixture of HS(C₃-C₃₀)alkyl-OH and HS(C₃-C₃₀)alkyl-CO₂H to form a self-assembled monolayer (SAM) covalently bonded to the surface of the noble metal via the sulfur moieties in the mixture;
b) bioconjugating pathogen-specific antigens of a virus envelope protein to —CO₂H moieties of SAM, thereby forming a working electrode; and
c) spacing a reference electrode adjacent to the working electrode thereby forming the micro-sensor;

wherein the micro-sensor is label-free and changes in electrical properties of the working electrode are detectable when an antibody binds with specificity to the antigen and forms an antigen-antibody complex.

In various other embodiments, the mole percent of HS(C₃-C₃₀)alkyl-OH is about 40% to about 60%, and the mole percent of HS(C₃-C₃₀)alkyl-CO₂H is about 40% to about 60%. In additional embodiments, the above method comprises chemically activating the —CO₂H moieties of SAM prior to bioconjugation and chemically passivating the chemically activated —CO₂H moieties that remain after bioconjugation. In other embodiments, the noble metal is gold and the method comprises etching the gold with a mineral base, peroxide, oxygen plasma, or a combination thereof.

In yet other embodiments, the pathogen-specific antigen of the virus envelope protein is an antigen from the envelope protein of Chikungunya virus, Zika virus, Dengue virus, Yellow fever virus, or West Nile virus. In some other embodiments, the working electrode and the reference electrode are both microwires having diameters of about 1 micrometer to about 100 micrometers (or 5 micrometers to about 150 micrometers), and the working electrode and reference electrode are spaced in parallel about 0.5 millimeters to about 2 millimeters (or about 0.1 millimeters to about 5 millimeters) apart and across a sample well.

Additionally, this disclosure provides a method for detecting antibodies comprising:
a) contacting a sample with a micro-sensor, wherein the micro-sensor comprises:
   i) a gold working electrode covalently bonded to sulfur atoms of a self-assembled monolayer (SAM), wherein the SAM comprises —(C₃-C₃₀)alkyl-chains substituted at one end with sulfur and functionalized at a terminal end with a functional group;
   ii) pathogen-specific antigens of a virus envelope protein bioconjugated to at least 10% of the functional groups of the SAM; and
   iii) a reference electrode; and
b) determining the presence or absence of a change in capacitance of the microsensor;

wherein the micro-sensor is label-free and changes in capacitance relative to the reference electrode are detectable when an antibody that is present in the sample binds with specificity to the antigen of the working electrode and forms an antigen-antibody complex.

In further embodiments, the pathogen-specific antigen of the virus envelope protein is an antigen from the envelope protein of Chikungunya virus, Zika virus, Dengue virus, Yellow fever virus, or West Nile virus. In other embodiments, the reference electrode is a Ag/AgCl electrode. In yet some further embodiments, the micro-sensor has a detection limit of about 1 antibody molecule to about 100 antibody molecules in a sample volume of about 10 microliters to about 100 microliters.

In other embodiments, the micro-sensor has a detection limit of less than 100 antibody molecules in a sample volume or less than 1 milliliter or a detection limit of less than 25 antibody molecules in a sample volume or less than 0.5 milliliters. In yet other embodiments, the detection limit is less than 80 antibody molecules, less than 60 antibody molecules, less than 40 antibody molecules, less than 20 antibody molecules, or less than 5 antibody molecules. In other embodiments the detection limit is based on a sample volume of less than 250 microliters, less than 150 microliters, less than 75 microliters, less than 50 microliters, or less than 25 microliters.

### Results and Discussion

Sensor Design and Principles.

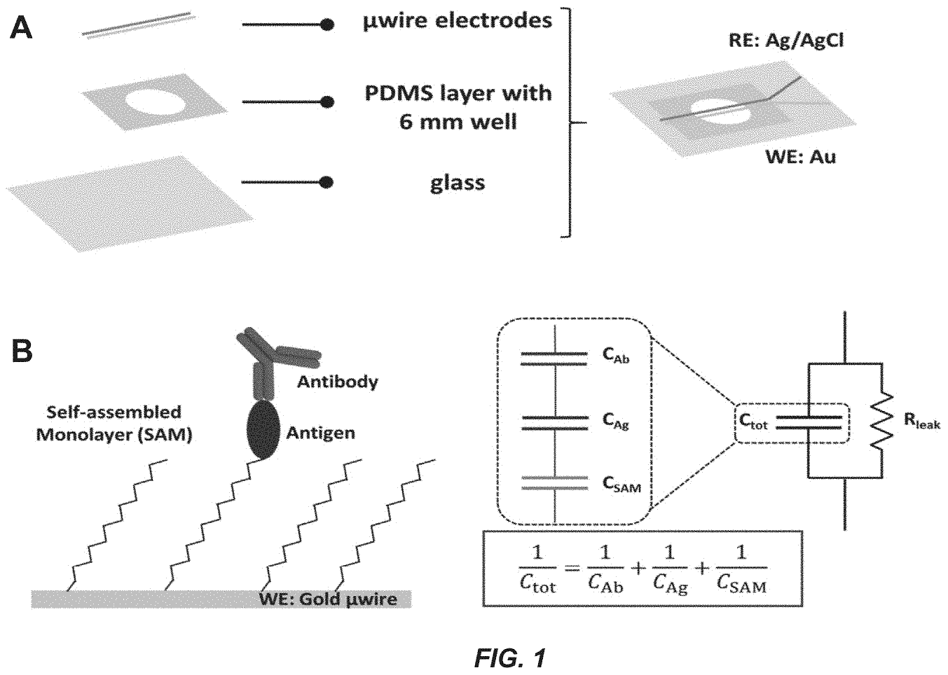

The label-free capacitive immunosensor introduced here uses microwire electrodes to rapidly and sensitively detect antibodies produced during an immune response, in this case mouse antibodies against ZIKV. The device is comprised of low-cost, easily acquired materials. A glass slide is used as the base substrate with a polydimethylsiloxane (PDMS) well for sample application. Au and Ag/AgCl microwires (working and reference electrodes, respectively) are immobilized across the PDMS well (FIG. 1a) and 30 μL of liquid sample is added to the well and incubated for 5 min. Measurements can then be taken in as quickly as one minute. Microelectrode wires, compared to other electrode fabrication methods like ink printing, paste, and sputter-coated electrodes, demonstrate increased mass transport rates due to radial diffusion. This increases the current density and consequently improves sensitivity and enhances detection limits. Microelectrodes offer the additional benefits of simple fabrication without expensive equipment, ease of surface chemical modification, and availability in different pure and alloyed compositions.

Randle's equivalent circuit is commonly employed to model the electrode-electrolyte interface of a Faradaic biosensor (Marks, 2013, Electrochemical Biosensors). However, our sensor has been designed as a non-Faradaic system to measure capacitive charging currents only. With no offset voltage applied to the electrode, off-target electrochemical reactions or charge transfer at the interface should be minimal. AC electrokinetic microflows have been known to affect capacitive charging currents, but these effects typically begin to occur at a peak-to-peak amplitude of 1 to 2 V and do not become prominent until 6 to 15 V. The influence of microflows at the 20 mV oscillation voltage used here is negligible. Thus, to model the charging current at the interface, we place C_{DL} in parallel with a leakage resistance, Rₗₑₐₖ. C_{DL} in turn can be modeled as the total capacitance, Cₜₒₜ, of several capacitors in series, as visualized in FIG. 1b. The first component constitutes the insulating SAM layer on the electrode surface, C_{SAM}. The second, C_{Ag}, includes the anchoring groups and the recognition element (antigen), which is followed by the concentration-dependent antibody layer, C_{Ab}.

Based on this model, the binding of antibody to antigen causes a change in the total capacitance, Cₜₒₜ. Because C_{SAM} is constant and does not contribute to capacitive change, the sensitivity of the sensor is predominately determined by the relative capacitance between antigen and antibody. In this case, use of a large analyte such as an antibody increases the sensitivity of our sensor by creating a proportionally larger increase in DL length compared to smaller analytes like antigens. This high sensitivity is necessary to adapt the immunosensor for pre-symptomatic pathogen detection, which is currently only achieved by nucleic acid testing.

Specificity Tests and Detection Limit.

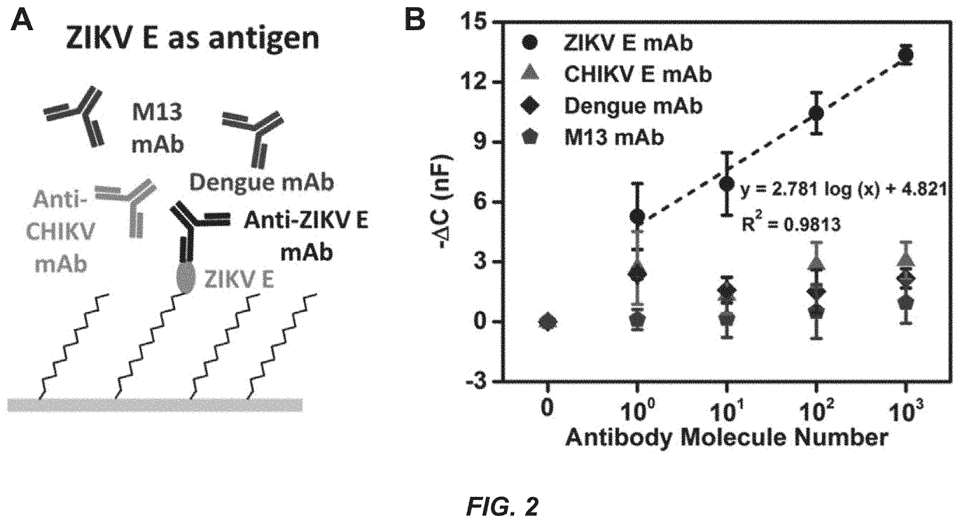

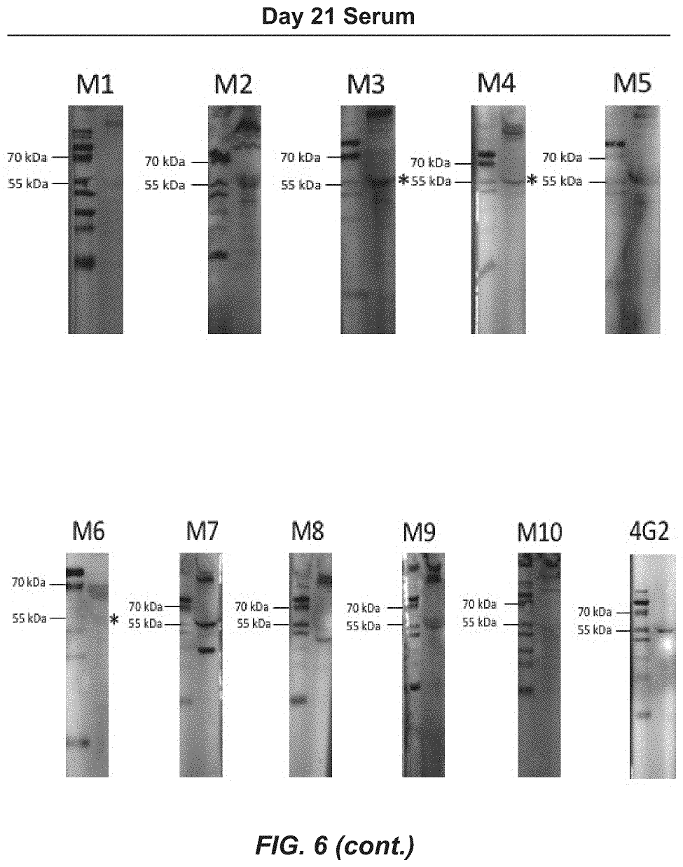

To characterize the immunosensor's performance, the ZIKV E-functionalized microwire sensor was first tested with monoclonal antibodies diluted in 1×PBST buffer (pH 7.4, 0.05% Tween 20). Anti-ZIKV E (experimental), anti-M13 antibody (control), anti-CHIKV E (control), and anti-DENV antibody (control) were tested (FIG. 2a). Each antibody was applied at concentrations ranging from ˜1 to ˜10³ molecules per 30 μL. The baseline capacitance reading (C_{Baseline}) after surface functionalization was directly recorded using an Instek LCR-821 benchtop LCR meter. Capacitance was again directly recorded after target antibody incubation (C_{Ab}). The mean negative capacitance change, −ΔC=−(C_{Ab}−C_{Baseline}), with standard deviation is presented in FIG. 2b for each sample (n=3). The −ΔC for anti-ZIKV E is proportional to the concentration/number of antibodies in the experimental sample and can be fit with a regression line (R²=0.9813). These results indicate proportionality between the magnitude of the capacitance change and the concentration of the bound target. In comparison, the −ΔC for controls have no significant change at any tested concentration, suggesting that there was no significant binding between ZIKV E and control antibodies. It is notable that the −ΔC for the ˜10 molecule anti-ZIKV E sample is statistically significantly different from the control antibody samples, indicating that the present detection platform has a detection limit as low as ˜10 antibody molecules per 30 μL, far superior to that of other immunosensors or ELISA assays. To demonstrate that the device can be adapted to other antigen/antibody pairs, the sensor was functionalized with CHIKV E2 antigen and tested with the same four monoclonal antibodies at the same concentration ranges (FIG. 7).

When normalized for baseline signal variations, the capacitance dropped ˜7% to 38% for the corresponding dynamic range. Collectively, these results show that the immunosensor functionalized with antigen can selectively capture antibodies at extremely low concentrations without nonspecific binding from other antibodies. This suggests an excellent combination of specificity and sensitivity for this platform. It is unclear what underlying mechanism gives rise to such significant signal changes at low concentrations, but the reported dynamic range was highly reproducible with different antigen-antibody pairs. It is well established that proteins randomly orient themselves when immobilized to a surface. As a result, binding regions of many probes are not accessible, leaving a portion of the surface inert and causing the active functionalized surface area to be much smaller than the total surface area. Therefore, while ˜10 antibody molecules may bind to only a small fraction of the total surface area, the proportion of the active surface area that is bound may be significantly larger and may contribute to large percentage changes in capacitance. Although significant advances have been made in the understanding of the interfacial region, thermodynamic models of functionalized surfaces fail when more complex charge distributions are considered. Further research is needed to elucidate what is happening at the interface of functionalized surfaces to understand the high sensitivity of our sensing system.

Isotyping Tests with Monoclonal Antibodies.

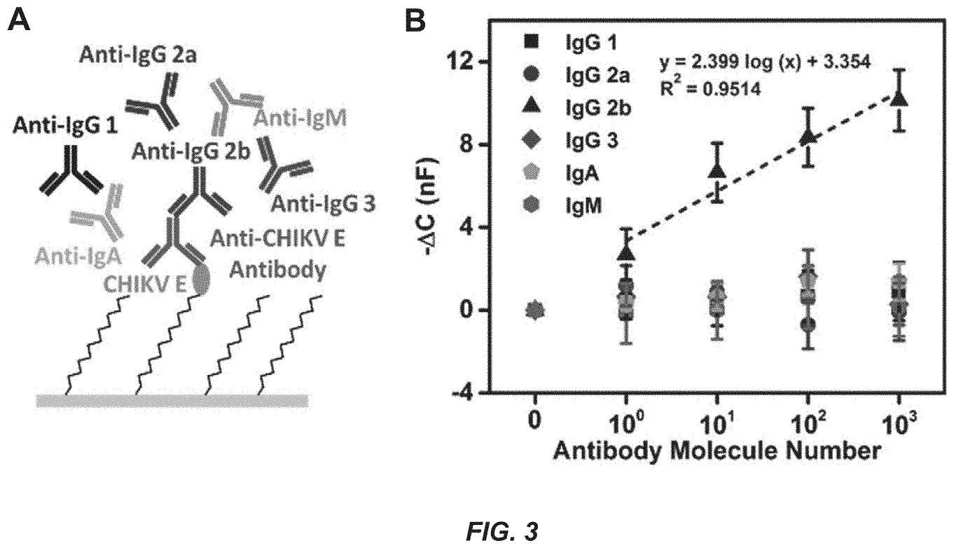

The isotype of antigen-specific antibodies is commonly determined to elucidate the stage of an infection, with IgM antibodies being present early in infection and IgG antibodies present later. To explore whether our platform could be used to determine isotype, the microwires were functionalized with CHIKV E antigen probe and subsequently saturated with corresponding IgG 2b antibody against CHIKV E (˜10³ molecule/30 μL). The capacitance value for anti-CHIKV antibody was set as a new baseline (C_{BL}). The devices were then incubated with six secondary antibodies with different specificities (IgG1, IgG2a, IgG2b, IgG3, IgA and IgM) at concentrations ranging from ˜1 to ˜10³ molecules per 30 μL (FIG. 3a). FIG. 3b presents the mean negative capacitance changes, −ΔC=−(C_{anti-iso Ab}−C_{Baseline}) with standard deviation for each sample (n=3). As predicted by the circuit model, an additional capacitance change was observed from anti-IgG2b antibody samples in all the concentrations applied. In addition, the −ΔC of anti-IgG2b antibody increases proportionally with increasing concentrations. In contrast, the five nonspecific anti-isotype antibodies did not increase the capacitance response (FIG. 3b). Supporting previous results of the detection limit, the capacitance of ˜10 anti-IgG 2b antibody molecules/30 μL is statistically significantly different from the nonspecific antibodies. These results indicate that our system can accurately determine the isotype of ultralow concentrations of antigen-specific antibodies.

Detection of Anti-ZIKV Antibodies During an Immunization Time-Course.

To explore the performance of the capacitive immunosensor in a complex matrix with interfering species, we tested mouse serum for ZIKV-specific polyclonal antibodies. Ten mice were immunized and samples were collected as described in the Examples. Mouse 3, 4, and 6 samples were tested with the ZIKV E functionalized sensor. Suitable dilutions were determined as described in the Examples.

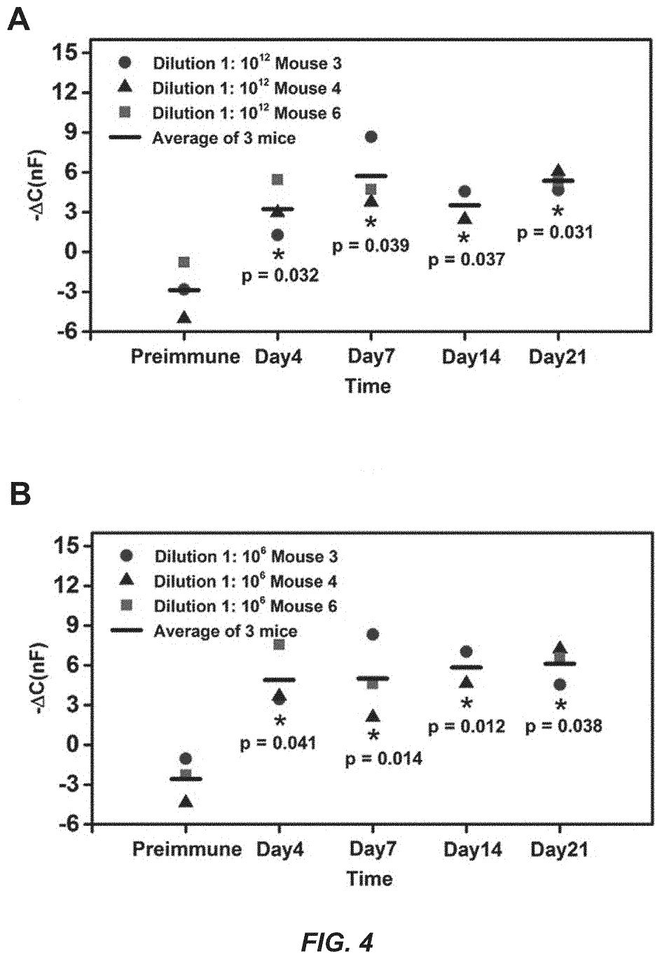

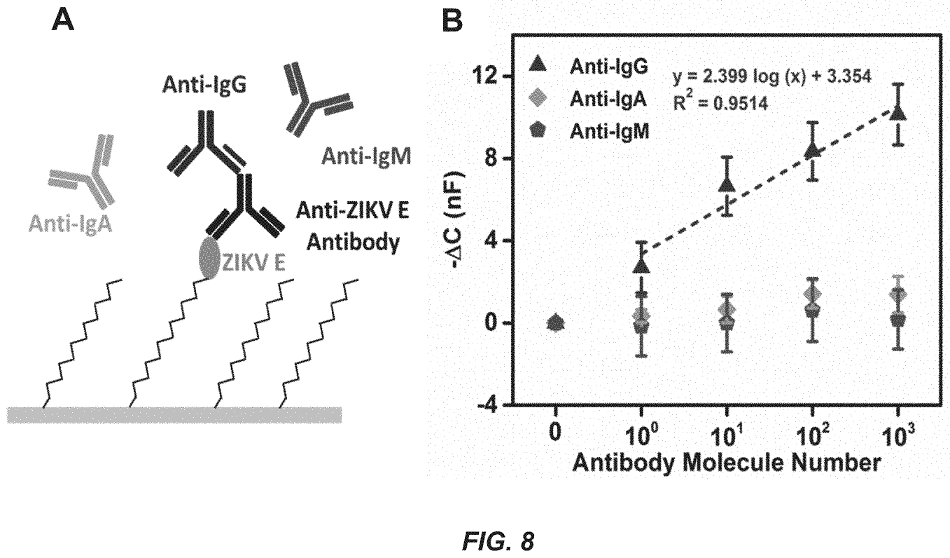

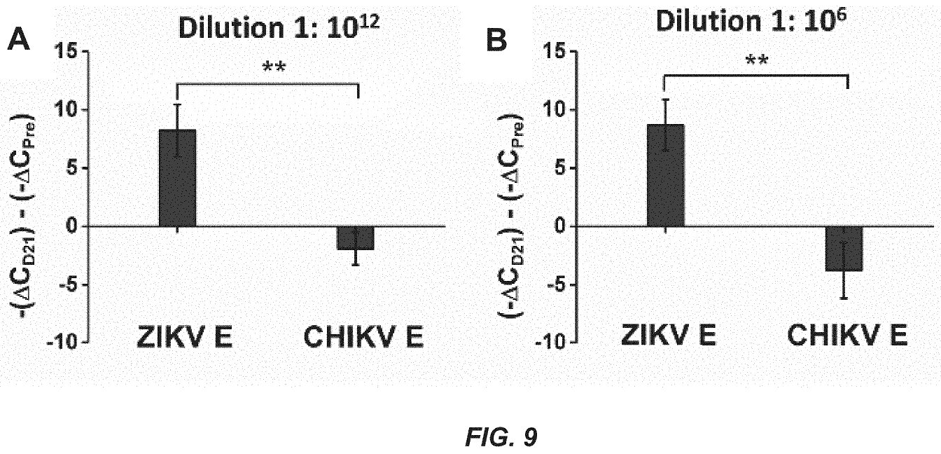

Based on the results in FIG. 10, two dilutions of the mouse serum, 1:10⁶ and 1:10¹² were chosen for detection for Day 4, 7, 14 and 21 mouse serum samples. Each of the three biological replicates was tested and averaged. Every biological replicate is the average of three technical replicates. The −ΔC for each post-vaccination sample was compared to the pre-immune sample as shown in FIG. 4a and FIG. 4b. At a 1:10¹² dilution, the −ΔC increases with each time point after vaccination and saturates around Day 14. The lower −ΔC for Day 14 can be attributed to its smaller sample size as there was no serum collected for mouse 6 on this day. Although results are similar for the 1:10⁶ dilution compared to the 1:10¹² dilution, it is notable that the −ΔC for this dilution saturates as early as Day 4 after immunization. Because the 1:10⁶ dilution is significantly more concentrated, this outcome is expected. More importantly, this capacitive immunosensor can detect extremely dilute antibody as early as four days post-vaccination through 21 days. To further characterize the specificity with mouse sera, we examined whether anti-ZIKV serum had any cross-reactivity with CHIKV sensors. The results described in the Examples (FIG. 9) show reproducibility of the sensor's specificity in a complex physiological matrix.

By reliably detecting as few as 10 molecules and accurately analyzing serum at dilutions of 1:10¹², the results suggest that our sensor has a far superior sensitivity compared to other platforms. This increased sensitivity enables us to detect an antibody response four days earlier compared to established serological methods. Our sensor also requires less sample volume than comparable ELISAs (30 μL of 1:10¹² vs 50-100 μL of 1:400 diluted sample (CDC 2016)), which preserves precious serum sample and reduces waste. Furthermore, whereas the CDC ZIKV MAC-ELISA needs 12+ hours to obtain results from sample application, our sensor can produce results in under ten minutes. This could result in faster diagnostics needed to determine a timely and effective therapeutic intervention.

Antibody Isotyping of Mouse Serum Samples.

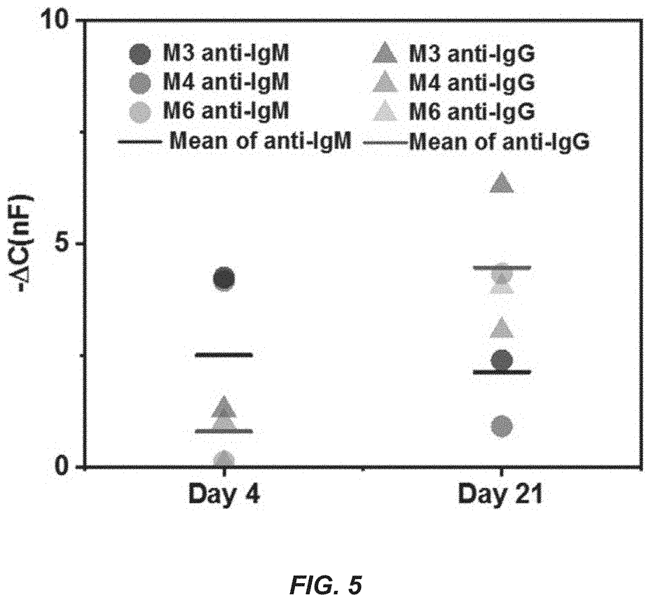

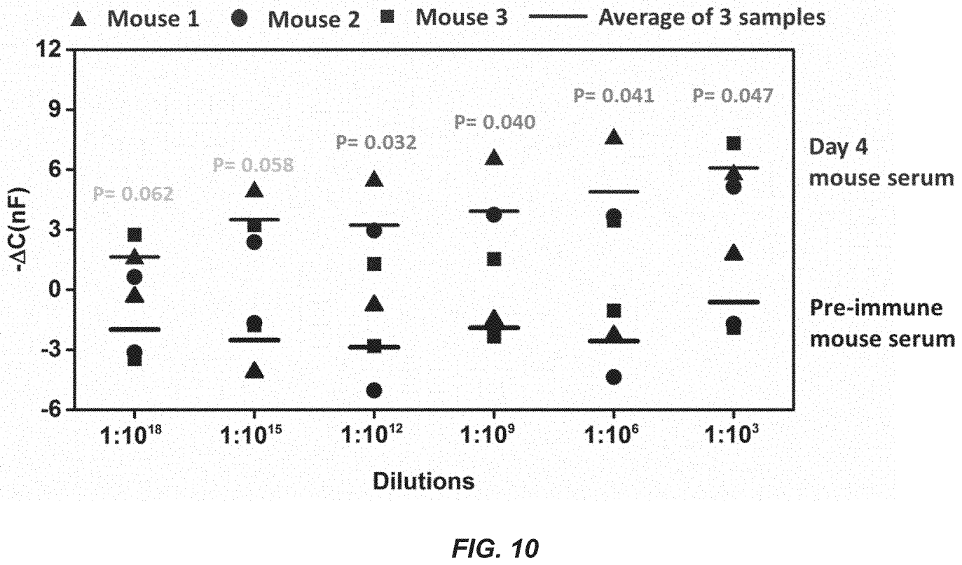

Antibody isotyping is a diagnostic component required to separate acute from past infections. To characterize whether our sensor platform can be used to determine the isotypes present in a serum sample, wire sensors were functionalized with ZIKV E protein and saturated with serum antibody from Day 4 or Day 21 at a 1:10⁶ dilution. Anti-mouse IgM or IgG was applied to the sensor and the results are compared in FIG. 5. As expected from published flavivirus antibody kinetics (Centers for Disease Control and Prevention, 2017) and the corresponding ELISA data (FIG. 11), Day 4 IgM levels were higher than IgG. It was somewhat surprising that the sensor detected constant levels of IgM between Day 4 and Day 21 given that the ELISA showed an increase from Day 4 to Day 21. This may be explained by saturation of the sensor. A recent report, however, indicates that anti-ZIKV IgM levels drop off 8 to 16 days after symptom onset. The discrepancy between our ELISA data and theirs may be due to our use of the immunodominant E protein instead of NS1 as antigen or it could be related to differences in host species. Antibody kinetics for dengue virus indicate that IgM can be detected for over 90 days (Centers for Disease Control and Prevention, 2017), suggesting that a higher titer for Day 21 is reasonable.

The sensor results also show an increase in IgG levels from Day 4 to Day 21, which agrees with the ELISA data. The sensor also shows higher IgG levels on Day 21 compared to IgM. These data conflict with the ELISA results, which show slightly higher IgM for both days. However, the ELISA assays in FIG. 9 were performed at 1:100 dilution, which was the highest dilution that gave detectable anti-ZIKV signals. We hypothesize that matrix effects in these concentrated mouse sera likely affected apparent antibody isotype distributions in the ELISA assays. Also, because the IgM is significantly larger than IgG, steric hindrance may cause the IgM sensor to saturate faster than the IgG sensor. As a smaller molecule, more IgG may be able to bind to the wire surface and produce a larger signal. Cabral-Miranda et al. recently published an immunosensor for ZIKV antibody with isotyping capacity that was able to detect a 10⁶ to 10⁷ dilution of serum (Biosens. Bioelectron. 2018, 113, 101). However, their design has decreased sensitivity compared to our system and it also incorporates a toxic redox couple that limits its POC use. Without using labels or redox couples, our sensor can distinguish antibody isotypes from a complex serum matrix containing a mixture of isotypes. These results enhance the applicability of the sensor for POC diagnosis and even for research purposes.

Conclusions.

Diagnosis of infectious diseases like ZIKV requires laboratory confirmation but current methodologies are limited to use by specialized diagnostic laboratories. Recent outbreaks like that of Ebola virus and ZIKV indicate a growing need for simple, sensitive, and selective diagnostics amenable to a POC setting. The ultra-sensitive capacitance sensor introduced in this study represents a simple and robust platform for antibody detection in serum. Within minutes, it can detect as few as ˜10 antibody molecules in a 30 μL volume and determine their isotype. Without using labels or redox couples, our sensor can detect anti-ZIKV antibodies during an immunization time course and distinguish the isotype from a complex serum matrix. Furthermore, this sensor design can be easily integrated with microfluidics and handheld measuring devices to make it suitable for field work and POC testing. This immunosensor platform can be integrated into our previously developed paper-based analytical device (Anal. Chem. 2018, 90, 7777). Continued development of this novel platform can greatly increase the capacity of public health agencies worldwide to assess drug or vaccine efficacy and to monitor emerging infectious diseases of global importance in future.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Material and Methods

Study Design.

The working microwire surface was functionalized with E protein from either ZIKV (ZIKV E) or Chikungunya virus (CHIKV E). Lower dynamic range boundaries for the device were first determined with monoclonal antibody samples. Anti-ZIKV E antibody was employed as a specific target while anti-CHIKV E, anti-Dengue, and anti-M13 were used as nonspecific targets. The microwire biosensor was also used to isotype the monoclonal antibodies with anti-mouse IgG1, IgG2a, IgG2b, IgG3, IgA and IgM antibodies. The microwire sensor was then validated using pre-immune and immune mouse sera collected 4, 7, 14 and 21 days post-ZIKV immunization. Next, the sensor was used to isotype Day 4 and 21 mouse sera for IgM and IgG.

Information for immunization and sera characterization, electrode functionalization and sensor fabrication are described in the Examples. Representative serum samples positive for ZIKV IgG antibody by Western blot were included in the serum testing. Control samples and experimental sample replicates are indicated in the text and figure legends.

Materials and Equipment.

Potassium hydroxide (KOH), iron (III) chloride hexahydrate (FeCl₃.6H₂O), 30% hydrogen peroxide (H₂O₂), and absolute ethanol were purchased from Fisher Scientific (Fairlawn, N.J.). High-purity silver ink was purchased from SPI Supplies (West Chester, Pa.). 11-Mercaptoundecanoic acid (MUA) was purchased from Santa Cruz Biotechnology (Dallas, Tex.). 3-Mercapto-1-propanol (MPOH) was purchased from Tokyo Chemical Industry Co., Ltd. (Portland, Oreg.). N-Hydroxysuccinimide (NHS) and 1-Ethyl-3-(3-dimethylaminopropyl)-carbodiimide (EDC) were purchased from Acros Organics (Geel, Belgium). Ethanolamine, Tween-20, and 2-(N-morpholino) ethanesulfonic acid (MES) was purchased from Sigma-Aldrich (St. Louis, Mo.). Phosphate buffered saline (1×PBS: 137 mM NaCl, 2.7 mM KCl, 10 mM Na₂HPO₄ and 1.8 mM KH₂PO₄, pH 7.4) was purchased from Hyclone (Logan, Utah). All reagents were used as received without further purification. All stock solutions were prepared using ultrapure water (18 MΩ cm) purified with the Nanopure System (Kirkland, Wash.). Wires of 99.99% pure gold (25 μm) and silver (25 μm) were purchased from California Fine Wire Company (Grover Beach, Calif.) and used as the working and reference electrode materials, respectively.

Recombinant ZIKV E, recombinant CHIKV E, and mouse monoclonal anti-CHIKV E antibodies were purchased from MyBioSource, Inc. (San Diego, Calif.) and stored at −20° C. until use. M13 antibody (Abcam ab24229), anti-dengue 2 envelope antibodies (Abcam ab80914), and ZV-2 Anti-Zika envelope antibody were generously provided by Dr. Michael Diamond. The concentration of ZIKV and CHIKV monoclonal antibodies was validated using a Nanodrop 2000c spectrophotometer from Thermo Scientific (Waltham, Mass.). ZIKV immune mouse serum was generated after DNA immunization of mice with ZIKV virus-like particle expression plasmids modeled from previous work (Virology 2006, 346, 53). Details for the construction of the immunization plasmids, immunization, serum collection, and initial antibody testing of serum are found in the Examples. Anti-Mouse IgG1, IgG2a, IgG2b, IgG3, IgG, IgA and IgM antibodies were purchased from BD Biosciences (San Jose, Calif.), and stored at 4° C. until use.

Capacitance Measurement Device and Setup.

The working electrode functionalization and sensor fabrication protocols are described in the Examples. Capacitance measurements were collected using an Instek LCR-821 benchtop LCR meter (New Taipei City, Taiwan) with a PC interface for data acquisition. Because DL capacitance is a non-Faradaic signal, a 0 V DC bias voltage was applied. A 20 mV root mean square (RMS) AC voltage was applied to the sensors at 20 Hz. All capacitance readouts were recorded in parallel mode in 30 μL of 0.1×PBST and 60 data points were collected per reading. A Faraday cage was used to remove electrical interference during readout. Capacitance data was analyzed using Matlab (Mathworks) and statistical tests were performed using R (www.r-project.org). Only p values less than 0.05 were considered statistically significant.

Monoclonal Antibody and Isotype Detection.

For all antibody detection studies, a 30 μL dilution of monoclonal antibody was added to the well and incubated for 5 min at room temperature in 1×PBST buffer containing ˜1 to −10³ molecules of each monoclonal antibody. The 5 min incubation time was selected by monitoring the rate of signal change; in all experiments, sufficient signal to noise was obtained. Additional discussion regarding incubation time may be found in the Examples. Following incubation, electrodes were rinsed three times first with 1×PBST buffer to remove residual protein and then again three times with 0.1×PBST buffer to remove excess salts that may interfere with electrochemical readout. For isotype determination, 30 μL of monoclonal CHIKV E antibody was added to the well and incubated for 5 min at room temperature in 1×PBST buffer. Electrodes were rinsed with 1× and 0.1×PBST, and antibodies against each isotype were added to the well at dilutions of −1 to −10³ in 1×PBST buffer. Electrodes were then rinsed again three times with 30 μL 1×PBST buffer and 30 μL 0.1×PBST buffer. Capacitance measurements were performed as described in the section 2.3.

Mouse Serum Sample Antibody and Isotype Detection.

Clarified mouse sera were diluted 1:10⁶ and 1:10¹² in 30 μL of 1×PBST buffer and incubated on microwire chips for 5 min at room temperature. Following incubation, electrodes were rinsed three times with 30 μL 1×PBST buffer and three times with 30 μL 0.1×PBST buffer. To determine the isotype of anti-ZIKV antibodies in the mouse sera the microwire sensor was first immersed in 30 μL of mouse serum diluted 1:10⁶ in 1×PBST for 5 min at room temperature. Antibodies specific for each isotype were then incubated for 5 min at dilutions of 1:10⁶ and 1:10¹² in 30 μL 1×PBST buffer. Following incubation, electrodes were rinsed three times each with 30 μL 1×PBST and 0.1×PBST buffer before measurements.

### Example 2. Electrode Functionalization and Sensor Fabrication

A 25 μm diameter Au microwire was used as the working electrode. To prepare the electrode surface, the Au microwire was immersed in a 20 mL solution of 50 mM KOH and 25% H₂O₂ for 10 min, and thoroughly rinsed in Milli-Q water to remove residual reagent. This widely used cleaning protocol removes debris that interferes with the stability of immobilized surface structures. The Au microwire was then plasma cleaned for 2 min in an 02 Plasma Etch PE-25 (Plasma Etch, Carson City, Nev., USA) at a pressure of 200 mTorr and with 150 W applied to the RF coil. A self-assembling monolayer (SAM) formation reaction was performed immediately after plasma cleaning which spontaneously forms an organized structure at the surface. Some SAM-forming molecules do not bind strongly to their substrate, like perylenetetracarboxylic dianhydride (PTCDA) on gold, and the resultant structures have poor stability. However, other molecules with stronger affinity such as alkanethiols, silanes, and phosphonate, have better stability.

In this study, alkanethiol chains were used to generate a more stable SAM. The thiol-metal bonds are on the order of 100 kJ/mol and are stable in a wide range of temperatures, solvents, and potentials. Briefly, a 10 mM mixed solution consisting of a 1:1 ratio of 3-MPOH (3-Mercapto-1-propanol) to 11-MUA (11-Mercaptoundecanoic acid) was prepared in the absolute ethanol. Ultraviolet (UV) radiation and variations in temperature and chemical environment have been shown to affect SAM stability and were controlled in this study to mitigate degradation of the SAM. The gold microwires were immersed in the mixed solution for 48 h without light at controlled room temperature and then rinsed three times with deionized water to remove residual reagent.

The MUA carboxyl groups were immediately activated for antigen coupling by a two-step NHS/EDC bioconjugation protocol. The SAM-modified gold microwires were incubated in 20 mL of 20 mM EDC and NHS in 0.1 M MES (2-(N-morpholino) ethanesulfonic acid) (pH 6.0) buffer for 30 min and then rinsed with 20 mL 0.1 M MES buffer. A solution of 8 μg/mL antigen (ZIKV E or CHIKV E) was incubated on the activated MUA surface for 2 h. After antigen incubation, the surface was incubated in 0.1 M ethanolamine in 1×PBS solution for 30 min to passivate unbound, activated MUA. The wire was rinsed with 1×PBS, incubated for 10 min, then rinsed three times with 30 μL of 0.1×PBS buffer before baseline measurements.

The sensor was constructed using a glass substrate with a 1 mm-thick polydimethylsiloxane (PDMS) layer, and two metal microwires. A 6 mm diameter hole in hydrophobic PDMS that was bound to the hydrophilic glass slide was used to contain liquid. To make the PDMS layer, PDMS prepolymer [RTV 615 A and B (10:1, w/w)] was mixed, degassed, then poured onto a flat silicon wafer to yield a 1 mm-thick fluidic layer. The PDMS layer was baked for 30 min at 80° C., then peeled from the silicon wafer. A biopsy punch (Technical Innovations, FL, Inc. USA) was used to create 6 mm diameter wells for sample containment. Both the PDMS and glass were exposed to oxygen plasma (Plasma Etch, NV, USA) for 1 min, then contacted to form a permanent bond.

On the PDMS with a 6 mm diameter well, Ag/AgCl and Au microwires were spaced 1 mm apart across the well. A two-electrode system was employed using Au and Ag/AgCl microwires as the working and reference electrodes, respectively, each with a surface area of 4.7×10⁻³ cm². The Ag/AgCl reference electrodes were made by dipping silver Ag wire in 50 mM iron (III) chloride for 50 s, forming a silver chloride layer on the surface. Silver paint was applied to wire ends to create touchpads that could be connected to the capacitance reader.

### Example 3. Preparation of Plasmids for Zika DNA Immunization

Genes for the Zika virus PRVABC59 strain (NCBI Accession: KX087101) capsid and prM-Env proteins were codon-optimized for mammalian expression and synthesized by Genescript Inc. The V5 epitope tagged capsid gene was cloned into the EcoRV site of pcDNA3.1 (plasmid pBG610), and a Japanese encephalitis virus prM signal sequence was added to the prM-Env gene and the construct was cloned into the EcoRV site of pcDNA3.1 (pBG611) as previous described (Virology 2006, 346, 53). Plasmid sequences will be provided upon request. Expression of capsid and prME proteins after transfection into Vero cells was verified by Western blot analysis using anti-V5 (Life Tech) and anti-Envelope (4G2 (ATCC HB-112 (D1-4G2-4-15))) antibodies, respectively.

DNA was prepared for immunization using the TempliPhi Rolling Circle Amplification Kit (GE HealthCare) according to the manufacturer's instructions. DNA was purified by phenol:chloroform extraction and ethanol precipitation, quantified by UV spectrometry, and stored at −20° C. until DNA immunization. Equal molar amounts of each amplified DNA were prepared in saline at 2 μg total DNA/50 μL or 10 μg total/50 μL prior to immunization.

### Example 4. DNA Immunization

Ten, 6-week old female CD1 outbred mice were purchased from Jackson Laboratories for use in DNA vaccination studies. Pre-immune sera were collected from each mouse via submandibular vein punctures, and five mice (Mice 1-5) were immunized intramuscularly with 4 μg total DNA (50 μL of 2m/50 μL in each flank (Mice 1-5)) or 20 μg total DNA (50 μL of 10 μg/50 μL in each flank (Mice 6-10)). A 100 μL whole blood sample was collected via retro-orbital bleed at Days 4, 7, 14, and 21 post-immunization. At Day 28 post-immunization, mice were anesthetized with isoflurane and terminal bleeds were collected via cardiac puncture. Sera was separated from whole blood via centrifugation at 13K RPM, and clarified sera were stored at −20° C. in single-use aliquots until use.

### Example 5. Incubation Time

For all experimental studies a 5 min target incubation time was used to ensure consistency among the different studies. This relatively short period was selected for two reasons: (1) a desired outcome of the project is an assay suitable for point of care, and time-to-answer is a key parameter; (2) preliminary studies showed that the rate of signal change dropped off after 5 min, with more than adequate signal to noise at the 5 min mark.

If one were to use the classic order of magnitude estimate for the time required to allow all target molecules to diffuse through a stationary, 30 μL liquid sample to the conjugated microwire, it would predict 40 min. The reason that only 5 min is needed to achieve a sufficiently quantifiable signal is likely due to the convection and recirculation patterns induced when liquid is pipetted into the sample well. The convective transport of target molecules significantly reduces or eliminates the mass transfer limitations inherent to many microarray applications.

### Example 6. Initial Assessment of IgG Antibody in Immunized Sera Via Western Blot and ELISA

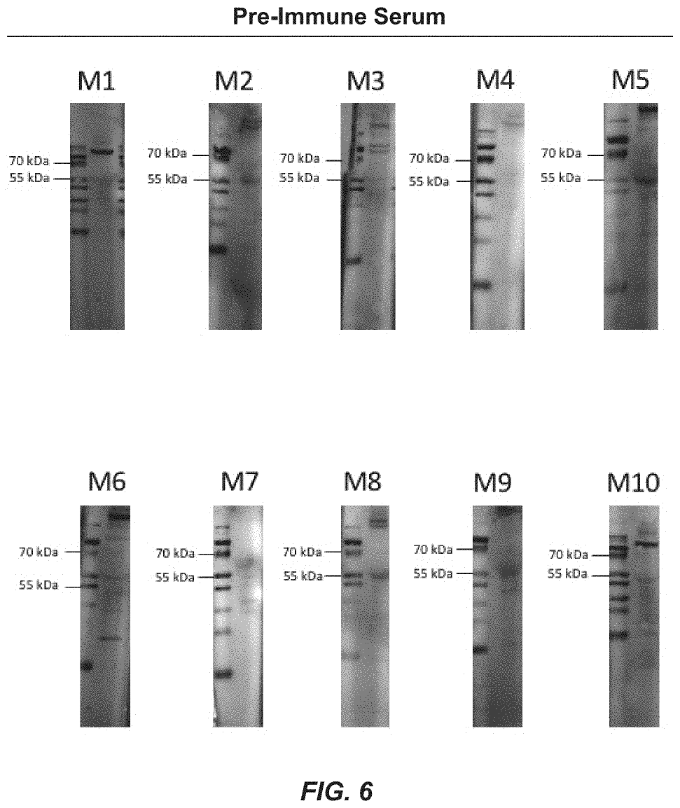

Aliquots of Day 21 sera from Mice 1-10 were used as the source of primary antibodies in strip Westerns. Vero cells (ATCC CCL-81) infected with the PRVABC59 strain of Zika virus were lysed in Laemelli buffer (Bio-Rad Cat #161-0737) and resolved on 12% PAGE gels. Proteins were transferred to nitrocellulose membranes that were subsequently cut into strips. Strips were blocked in phosphate-buffered saline 0.05% Tween+2% non-fat dried milk (Carnation Brand) (PBST-NFDM), then incubated in PBST-NFDM with each sera (1:100 dilution) overnight. 10 μg/mL of 4G2 antibody was used as a control. Strips were washed with PBST, incubated with anti-mouse IgG HRP (abcam # ab6728) in PBST-NFDM for 1 h, washed with PBST, and developed with Pierce 1-step Ultra TMB-blotting solution. Based on the results of the Western blots shown in FIG. 6, sera from mouse 3, 4, and 6 were chosen for biosensor analysis. The DNA vaccine comprised of two expression plasmids, one containing a sequence for the capsid (C) protein, the other containing the sequence for pre-membrane and envelope proteins (prM-E), which spontaneously assemble in the cell to form subviral, non-infectious particles. Subviral particles are smaller than their infectious counterparts which increases the curvature of the membrane and alters the icosahedral arrangement of E protein. These differences could affect access to epitopes which may be responsible for the low immunogenic success (30%) of the DNA vaccine.

### Example 7. Sensor Adaptability

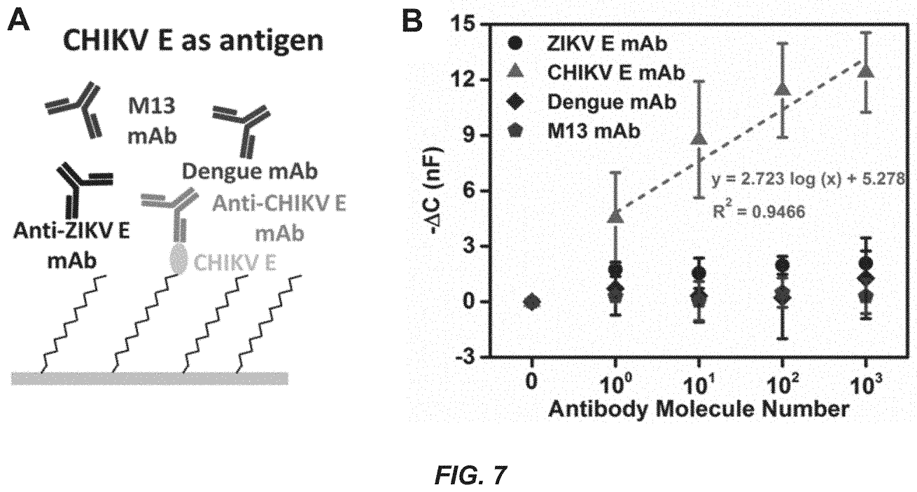

To demonstrate that the device can be adapted to other antigen/antibody pairs, the sensor was functionalized with CHIKV E2 antigen and tested with the same four monoclonal antibodies at the same concentration ranges. As expected, the −ΔC obtained from anti-CHIKV E antibody is proportional to the concentration/number of corresponding anti-CHIKV E antibody and is fitted with a regression shown in FIG. 7 (R²=0.9466). The other three nonspecific antibodies did not induce significant responses. Again, the −ΔC obtained from anti-CHIKV E antibody sample containing 10 molecules is statistically significantly different from the three non-specific antibodies, which confirms a detection limit of ˜10 antibody molecules/30 μL. Isotyping of anti-ZIKV monoclonal antibody was performed as described in the main text for anti-CHIKV monoclonal antibody, with the modification of anti-IgG, anti-IgA, and anti-IgM being tested. Anti-ZIKV E antibody showed a similar response as anti-CHIKV antibody (FIG. 8).

### Example 8. Specificity Tests with Mouse Serum

CHIKV E antigen was conjugated to the microwire as a control probe to test two dilutions (1:10¹² and 1:10⁶) of the pre-immune and ZIKV-vaccinated Day 21 mouse serum. FIG. 9 compares the −ΔC results obtained with specific ZIKV E probe and control CHIKV E probe. The y-axis marks the difference in −ΔC between Day 21 and pre-immune samples, and the x-axis denotes the two probes used. As shown in FIG. 9a, −ΔC between Day 21 and pre-immune mouse serum using ZIKV E probe is approximately 9 nF at the 1:10¹² dilution, suggesting that ZIKV antibody concentrations increase significantly after 21 days post vaccination. In comparison, the CHIKV E sensor shows almost no change (˜0 nF), 21 days post ZIKV vaccination, indicating that only specific binding occurred. A small increase in capacitance may be attributed to small amounts of nonspecific adsorption. There is a statistically significant difference between the ZIKV E and CHIKV E functionalized sensors. Similar results are observed for a 1:10⁶ dilution (FIG. 9b).

These results demonstrate satisfactory reproducibility and further validate the excellent specificity and sensitivity of this platform in a complex physiological matrix. Therefore, our sensor may be useful for direct detection of antigen-specific antibodies in serum and other potential types of biological sample.

### Example 9. Dilution Range

To determine suitable dilutions of the mouse serum samples for the platform, the pre-immune and Day 4 mouse sera were tested with a wide range of concentrations (1:10¹⁸ to 1:10³ dilutions in 1×PBST). As shown in FIG. 10, the average −ΔC obtained from the Day 4 serum increases along with increased concentration and the pre-immune sera conversely shows no significant change in the average −ΔC across the dilution range. There is no significant difference between pre-immune and Day 4 serum at dilutions lower than 1:10¹². All dilutions at and above 1:10¹² show statistically significant differences with p-values less than 0.05 (FIG. 10). These results indicate that 1:10¹² is the highest dilution that can be used with these serum samples to detect statistical differences. Therefore, this platform can differentiate vaccinated from non-vaccinated mouse serum at ultra-dilute concentrations as low as 1:10¹² and as few as four days after vaccination. This is comparable to the early acute phase of infection before or concurrent with disease symptomology. Subsequently, this assay can extend the window of antibody detection into the early acute phase of infection.

### Example 10. ELISA Analysis of Anti-Zika IgM and IgG Levels in Mice Sera

An ELISA assay was used to determine the relative amounts of IgM and IgG in the Mouse 3, 4, and 6 Day 4 and Day 21 serum samples. Briefly, 100 μL of 10 μg/mL ZIKV E protein (My Biosource Cat # MBS319787) diluted in PBS (pH 7.4) was added to each well of a Nunc Maxisorp 96 well plate (Cat #44-2404-21) and incubated at 4° C. overnight. Excess antigen was discarded, and the wells were washed three times with 0.05% PBST (pH 7.4). 300 μL of fresh blocking buffer (4% milk powder in PBS) was then incubated in each well for 1 h at room temperature. Afterwards, the wells were washed six times with 0.05% PBST. A 100 μL mouse serum sample was then incubated for 1 h at room temperature at 1:100 dilution. 10 μg/mL of 4G2 antibody was used as a positive control. The wells were washed again six times with 300 μL of 0.05% PBST and 100 μL of 1:3000 HRP-conjugated anti-mouse IgG (AbCam ab97023) or IgM (AbCam ab97230) was incubated for 1 h at room temperature. The plate was washed six times with 300 μL 0.05% PBST then again twice with 300 μL of PBS to eliminate residual detergent. 100 μL of TMB-ELISA substrate (ThermoScientific) was incubated for 30 min at room temperature and quenched with 100 μL of H₂SO₄. Absorbance was measured at 450 nm. Results of the ELISA assay are shown in FIG. 11.

### Example 11. Validation of Immobilization Chemistries

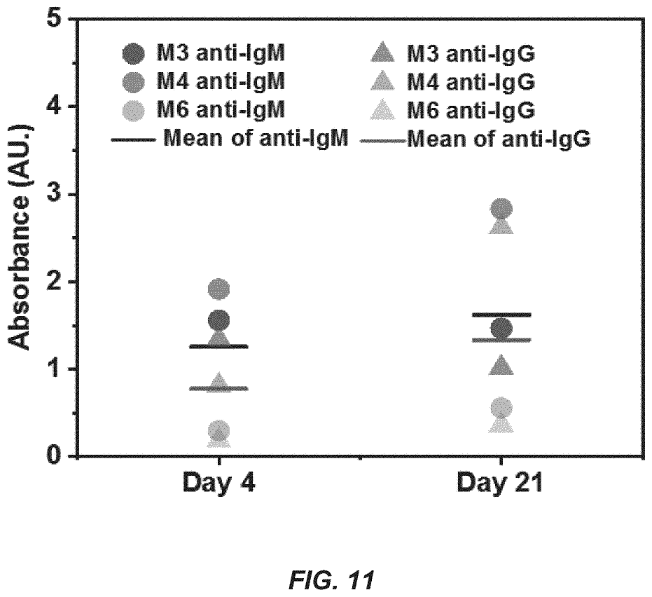

The ePAD is designed using Auto CAD software and fabricated by a standard wax patterning method. As shown in FIG. 1a, on the wax-printed side of paper, Ag/AgCl and Au microwires were spaced 1 mm apart across the device. The gold microwire serves as the working electrode, which is first treated with a self-assembled monolayer (SAM), then bound with recombinant ZIKV envelope protein (E) as the capture probe. Ethanolamine (ETA) is used to block the uncovered gold surface. Once the ePAD has been fabricated, a specific target-ZIKV E monoclonal antibody (mAb) and different nonspecific antibodies (anti-M13 mAb; antidengue-E mAb and anti-chikungunya-E mAb), are separately introduced onto the ePAD to measure their impedance/capacitance signals. A schematic of the processes mentioned above are shown in FIG. 12. The paired t-test is used for statistical analysis and p-values less than 0.05 are deemed as statistically significant.

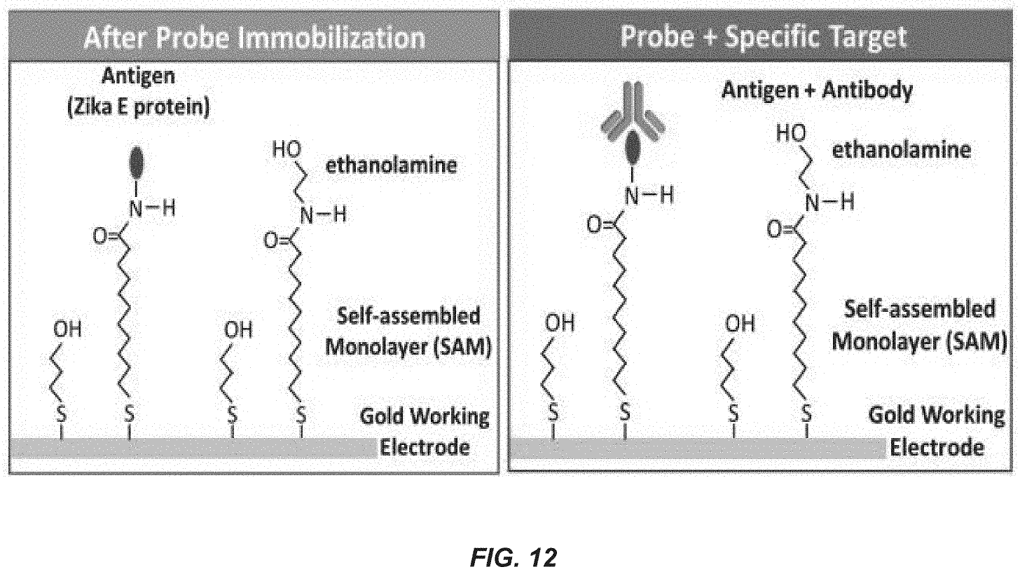

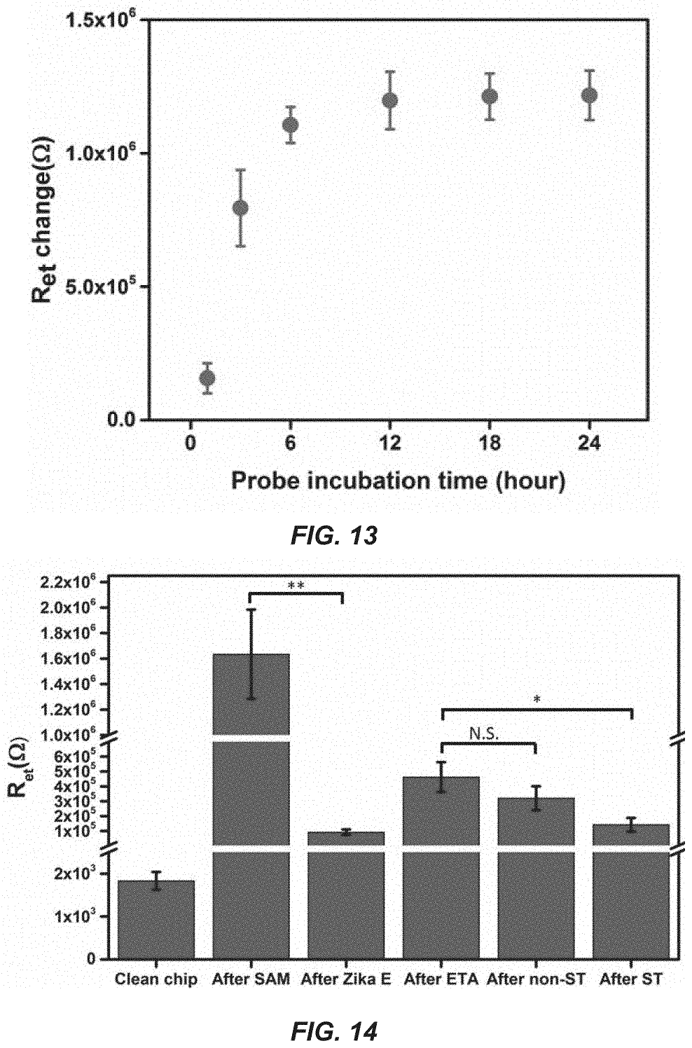

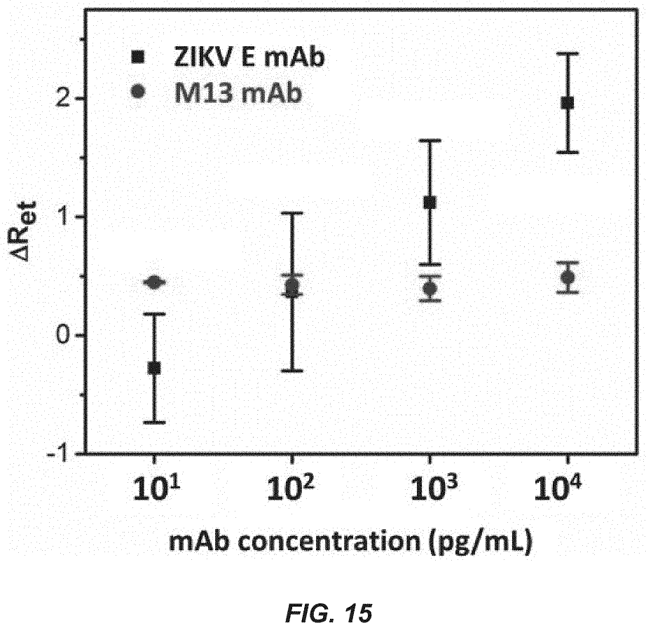

A label-free immunoassay ePAD using microwire electrodes has been developed and tested for ZIKV antibody detection. Initial studies were focused on choices of probes to achieve favorable sensitivity and specificity. ZIKV E protein was chosen because it is recognized by neutralizing antibodies. Then the probe immobilization strategy was improved as shown in FIG. 13. Once the probe immobilization chemistries were validated (FIG. 14), measurements were carried out using the specific ZIKV E mAb and nonspecific M13 mAb. As shown in FIG. 13, binding between antigen and specific target results in a significant decrease in resistance, while the change for the non-specific mAb is small. The quantitative response to specific and nonspecific target are shown in FIG. 15. The magnitude of electron transfer resistance increases with specific target concentration between 10 pg/mL and 10 ng/mL.

The biosensor has the ability to detect specific antigen-antibody with a detection limit down to 10 antibody molecules, with a dynamic linear range of detection from 1×10\hat{ }1 to 1×10\hat{ }3 antibody molecules.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A micro-sensor comprising:
a) a working electrode covalently bonded to head-groups of a self-assembled monolayer (SAM), wherein the SAM comprises alkyl chains, wherein the alkyl chains are substituted at one end with a head-group and functionalized at a terminal end with a functional group; and
b) pathogen-specific antigens bioconjugated to at least 10% of the functional groups of the SAM;
wherein the micro-sensor is label-free and changes in electrical properties of the working electrode are detectable when an antibody binds with specificity to the antigen and forms an antigen-antibody complex.

2. The micro-sensor of claim 1 wherein the pathogen-specific antigen comprises an antigen of a virus envelope protein.

3. The micro-sensor of claim 1 wherein the pathogen-specific antigen comprises an antigen of a flavivirus envelope protein or an alphavirus envelope protein.

4. The micro-sensor of claim 1 wherein the pathogen-specific antigen comprises an antigen of envelope proteins of Chikungunya virus, Zika virus, Dengue virus, Yellow fever virus, or West Nile virus.

5. The micro-sensor of claim 1 wherein the pathogen-specific antigen is bioconjugated to the functional groups via an amide bond.

6. The micro-sensor of claim 1 wherein the SAM comprises a second functional group comprising a hydroxyl.

7. The micro-sensor of claim 1 wherein the alkyl chains comprise —(C₃-C₃₀)alkyl-.

8. The micro-sensor of claim 1 wherein the head groups comprise sulfur, silicon, or phosphorous.

9. The micro-sensor of claim 1 wherein the working electrode comprises an oxygen plasma etched noble metal.

10. The micro-sensor of claim 1 comprising a silver microwire reference electrode, wherein the working electrode comprises a gold microwire, the alkyl chains are —S(C₃-C₃₀)alkyl-X covalently bonded to the gold microwire via the sulfur atom of —S(C₃-C₃₀)alkyl-X, wherein X of about 30% to about 80% of the alkyl chains is a bioconjugated pathogen-specific antigen of flavivirus envelope protein, and X of the remaining percentage of the alkyl chains is a functional group comprising hydroxyl, carboxyl, or amide.

11. A method for forming a micro-sensor comprising:
a) contacting a noble metal and a mixture of HS(C₃-C₃₀)alkyl-OH and HS(C₃-C₃₀)alkyl-CO₂H to form a self-assembled monolayer (SAM) covalently bonded to the surface of the noble metal via the sulfur moieties in the mixture;
b) bioconjugating pathogen-specific antigens of a virus envelope protein to —CO₂H moieties of SAM, thereby forming a working electrode; and
c) spacing a reference electrode adjacent to the working electrode thereby forming the micro-sensor;
wherein the micro-sensor is label-free and changes in electrical properties of the working electrode are detectable when an antibody binds with specificity to the antigen and forms an antigen-antibody complex.

12. The method of claim 11 wherein the mole percent of HS(C₃-C₃₀)alkyl-OH is about 40% to about 60%, and the mole percent of HS(C₃-C₃₀)alkyl-CO₂H is about 40% to about 60%.

13. The method of claim 11 comprising chemically activating the —CO₂H moieties of SAM prior to bioconjugation and chemically passivating the chemically activated —CO₂H moieties that remain after bioconjugation.

14. The method of claim 11 wherein the noble metal is gold and the method comprises etching the gold with a mineral base, peroxide, oxygen plasma, or a combination thereof.

15. The method of claim 11 wherein the pathogen-specific antigen of the virus envelope protein is an antigen from the envelope protein of Chikungunya virus, Zika virus, Dengue virus, Yellow fever virus, or West Nile virus.

16. The method of claim 11 wherein the working electrode and the reference electrode are both microwires having diameters of about 1 micrometer to about 100 micrometers, and the working electrode and reference electrode are spaced in parallel about 0.5 millimeters to about 2 millimeters apart and across a sample well.

17. A method for detecting antibodies comprising:
a) contacting a sample with a micro-sensor, wherein the micro-sensor comprises:
i) a gold working electrode covalently bonded to sulfur atoms of a self-assembled monolayer (SAM), wherein the SAM comprises —(C₃-C₃₀)alkyl-chains substituted at one end with sulfur and functionalized at a terminal end with a functional group;
ii) pathogen-specific antigens of a virus envelope protein bioconjugated to at least 10% of the functional groups of the SAM; and
iii) a reference electrode; and
b) determining the presence or absence of a change in capacitance of the microsensor;
wherein the micro-sensor is label-free and changes in capacitance relative to the reference electrode are detectable when an antibody that is present in the sample binds with specificity to the antigen of the working electrode and forms an antigen-antibody complex.

18. The method of claim 17 wherein the pathogen-specific antigen of the virus envelope protein is an antigen from the envelope protein of Chikungunya virus, Zika virus, Dengue virus, Yellow fever virus, or West Nile virus.

19. The method of claim 17 wherein the reference electrode is a Ag/AgCl electrode.

20. The method of claim 17 wherein the micro-sensor has a detection limit of about 1 antibody molecule to about 100 antibody molecules in a sample volume of about 10 microliters to about 100 microliters.
